(19) United States
(12) Patent Application Publication
(10) Pub. No.: US 2006/0264863 A1
(43) Pub. Date: Nov. 23, 2006

(54) One piece fitted disposable super absorbent article

(75) Inventor: Peter Compton Blyth, Valrico, FL (US)

Correspondence Address: INNOVATIVE TECHNOLOGIES GROUP, INC., 1002 FOXDALE PLACE, VALRICO, FL, 33594, US

(73) Assignee: Innovative Technologies Group, Inc., Valrico, FL (US)

(21) Appl. No.: 11/133,185

(22) Filed: May 20, 2005

(51) Int. Cl. A61F 13/15

(52) U.S. Cl. 604/393

(57) ABSTRACT

Disclosed is a one piece disposable, biodegradable, form fitting and absorbent protective article fabricated with a biodegradable non-woven hydrophilic top sheet, a fluid acquisition and distribution layer comprised of SAP, SAP/thermoplastic fiber blends or SAP/cellulosic fiber blends, a back sheet of a biodegradable fluid impervious thermoplastic film, and a contour conforming side panel capable of stretching both transversely and longitudinally and is secured with elastic banding or draw string. The entire article is fabricated by means of heat sealed contact points and hot melt adhesives.

### CROSS REFERENCE TO RELATED APPLICATIONS

### STATEMENT REGARDING FEDERALLY SPONSORED RESEARCH AND DEVELOPMENT

Not applicable

### FIELD OF INVENTION

This invention relates generally to a low cost, one piece, highly absorbent, disposable, form fitting cover, and more particularly, the present invention is directed to a biodegradable, super efficient SAP filled, disposable pad with associated extensible non-woven side panels and associated elastic banding or draw strings.

### BACKGROUND ART

U.S. Pat. No. 4,358,865 discloses a roll of absorbent disposable tear sheets that do not contain super efficient SAP nor are fabricated with non-woven materials.

U.S. Pat. No. 4,704,753 discloses a fabricated absorbent sheet with folded corners and fitted with elastic but without utilizing super efficient SAP materials.

U.S. Pat. No. 4,744,118 discloses a fitted extensible elastic banded bed sheet without absorbent materials.

U.S. Pat. No. 4,809,375 discloses a reusable pad with zippers, but is not form fitting, disposable nor uses super efficient SAP materials.

U.S. Pat. No. 4,961,982 discloses a reusable structured absorbent pad that is not fitted nor does it contain super efficient SAP.

U.S. Pat. No. 5,081,729 discloses a tape secured fitted disposable pad that utilizes an inferior absorbent material other than super efficient SAP.

U.S. Pat. No. 5,086,530 discloses a non-fitted detachable, washable reusable pad that utilizes an inferior absorbent material other than super efficient SAP.

U.S. Pat. No. 5,330,817 discloses a reusable and washable pad that utilizes an inferior 3-ply absorbent technology other than super efficient SAP.

U.S. Pat. No. 5,615,425 discloses a fitted disposable sheet without super efficient SAP materials.

U.S. Pat. No. 5,701,617 discloses a detachable absorbent center section attached to washable fitted side panels without utilizing super efficient SAP absorbent material.

U.S. Pat. No. 6,233,762 discloses an absorbent center panel with tucked fittings and no elastic nor any super efficient SAP absorbent material.

This invention solves the existing problems associated with absorbent pad articles. All competitive items have one or more of the following problems; (1) are multi component, (2) are reusable only if washed, with its attendant potential for retaining residual contaminants, (3) use inefficient absorbent materials leading to poor fluid acquisition and distribution and resultant poor skin dryness, (4) are not fitted with elastic banding or draw strings to ensure conformity to the surface to be protected, (5) provide inadequate surface coverage and (6) are not biodegradable, therefore not disposable in an environmentally sound manner. The disclosed article is a one piece, economical, biodegradable, disposable, efficiently absorbent pad fitted with extensible side panels and self-securing elastic banding or draw strings.

### DESCRIPTION OF THE PREFERRED EMBODIMENT

Broadly speaking, the present invention is a single-component article comprised of a non-woven fluid permeable top sheet, a SAP containing core designed to maximize fluid acquisition, distribution and retention and a fluid impervious blown film back sheet. The entire absorbent unit is further fabricated with extensible side sections permanently attached to the outside perimeter of the absorbent section and contour conforming elastic banding or draw strings attached to the outside perimeter of the extensible side panels.

In one embodiment, the absorbent pad area will be comprised of SAP or a blend of SAP and cellulosic fiber encapsulated between a fluid permeable non-woven top layer and fluid impermeable blow film bottom layer.

In one preferred embodiment, the top fluid acquisition layer will be comprised of non-woven textiles designed to maximize fluid acquisition in the least amount of time whether for initial surges or repeated gushes and to wick the acquired fluid to the fluid retentive layer as efficiently as possible to minimize the sensation of wetness on the surface.

In one preferred embodiment, the fluid retentive area will be comprised of encapsulated SAP in a variety of forms to include but not limited to powders, fibers or SAP in a matrix, whether the SAP is fixed to or freely dispersed amongst other elements such as cellulosic or other thermoplastic fibers.

In one embodiment, the bottom layer will be of sufficient size to extend beyond the dimensions of the permeable top layer area. The perimeter of the bottom layer will be folded up and over the outside perimeter of the top layer and sealed to the top layer utilizing techniques commonly known to those versed in the art forming a leak free fluid retentive perimeter.

In one embodiment, the outside perimeter of the fluid retentive portion of the pad is attached to an extensible non-woven skirt of sufficient size so as to conform to the dimensions of the target area needing protection from damaging fluids.

In one embodiment, the extensible skirt will utilize banding attachment techniques commonly known to those skilled in the art, which include, but are not limited to stitching, gluing, heat sealing and encapsulating.

In one preferred embodiment, the outside perimeter of the extensible skirt will utilize elastic banding to ensure size conformity and secure attachment.

In one preferred embodiment, the outside perimeter of the extensible skirt will utilize a draw string to ensure size conformity and secure attachment.

In one preferred embodiment, the outer perimeter of the side panel will be folded under itself to form a channel with the elastic band or draw string encapsulated within the channel by means currently utilized by those skilled in the art.

In one preferred embodiment, the article will be fabricated with fabrics, films and absorbents containing recycled content materials.

In one preferred embodiment, the absorbent article will be fabricated with hypoallergenic materials to minimize adverse allergic reactions.

In one preferred embodiment, the absorbent article will be fabricated with unbleached and chlorine-free cellulosic materials when cellulosics are utilized.

### BRIEF DESCRIPTION OF THE DRAWINGS (APPENDIX “a”)

FIG. 1. is an enlarged sectional view of the construction of the current article.

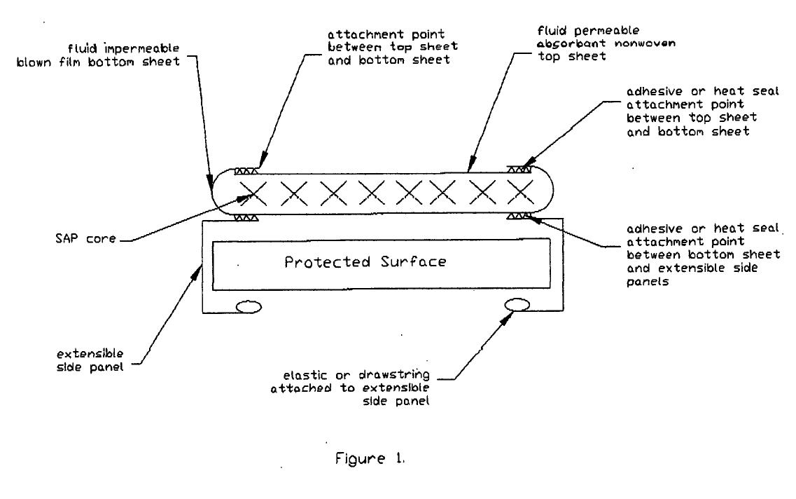

FIG. 2. is a side sectional view, along with a blown up view, of the absorbent top pad and the extensible side panel conforming to a surface that requires protection.

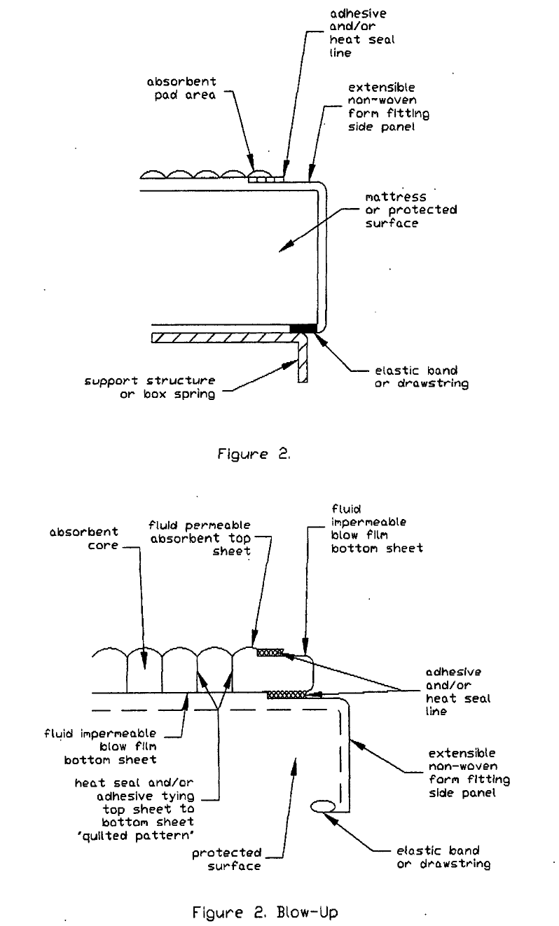

FIG. 3. is a partial set of examples of various surfaces that can be protected by the current article.

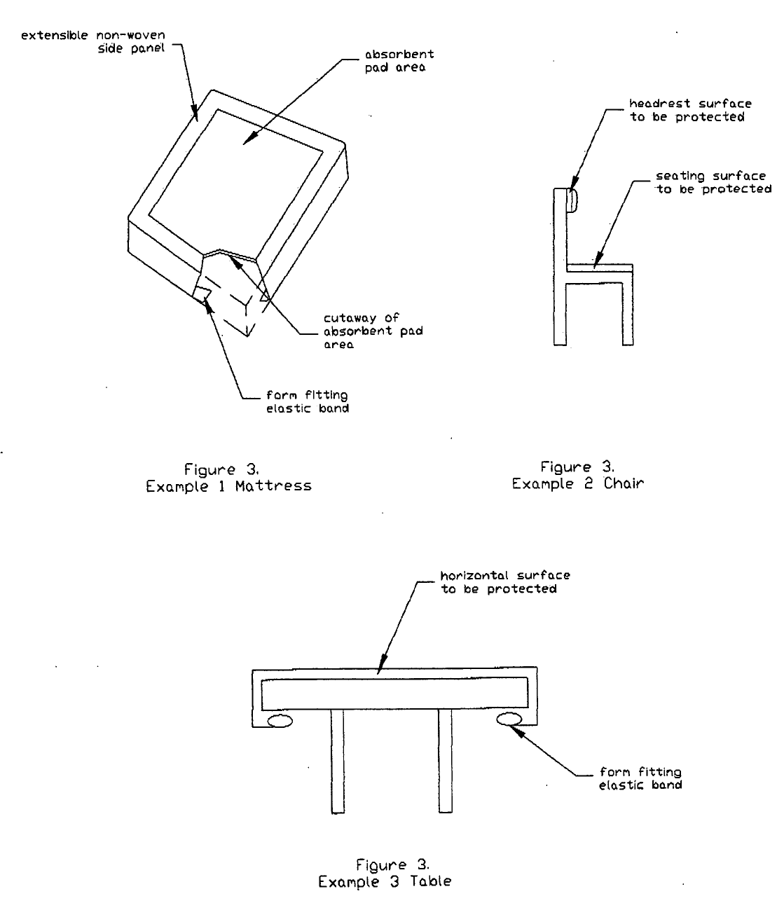

### DETAILED DESCRIPTION OF THE INVENTION

The present invention is directed to a form fitting absorbent protective cover for use as a personal or commercial hygienic article. This cover is designed and manufactured to be inexpensive and biodegradable thereby promoting ease of use and disposability. The cover is intended to be placed between the user and any surface requiring protection.

The protective cover is composed of a thermoplastic non-woven top sheet, a super absorbent core positioned over the entire surface to be protected all backed by a thermoplastic fluid impervious back sheet. The perimeter of the absorbent section of the cover will be joined by various mechanical means known to those versed in the art to extensible side panels with attached elastic banding or draw string which will secure the entire assembly to the surface requiring protection.

The top sheet will be joined at its outermost perimeter to the back sheet in such a manner as to encapsulate the super absorbent materials. The back sheet will be sufficiently larger in width and length than the top sheet so as to allow the back sheet to be folded up and back over the outside perimeter of the top sheet and then attached to the top sheet. The attachment techniques will provide for a continuous impervious joint which forms a fluid retentive pocket. The super absorbent containing center section preferable extends across the width and length of the desired surface to be protected although it could also be designed to cover less than the entire area that could potentially be protected. Preferable the super absorbent containing center section will be fabricated utilizing recycled content containing, biodegradable, hypoallergenic materials free of bleached, chlorine containing cellulosic materials.

With many potential surfaces requiring protection, the cover sheet and conforming side panels can be manufactured in a wide variety of dimensions. To ensure the protective absorbent cover remains in place, the extensible side panels and retentive elastic side bands or draw strings may also be constructed so as to conform to the surface of any size area requiring protection.

The utility of said article is a direct result of its ease of disposability and super absorbent nature while being constructed of biodegradable, hypoallergenic, chlorine-free, inexpensive thermoplastic materials. By manufacturing the devise in multiple sizes and dimensions, the user will find the products adaptability to be superior to other absorbent products/patents.

1. A one piece disposable protective article comprised of:
a fluid permeable top sheet,
an absorbent core comprising super absorbent polymers,
a fluid impervious back sheet, and
an extensible side panel attached to the said fluid impervious back sheet.

2. A protective article as described in claim # 1, wherein said side panel attaches with elastic draw string or banding for self-securing the entire assembly to the surface to be protected.

3. (canceled)

4. (canceled)

5. (canceled)

6. A protective article as described in claim # 1, wherein said absorbent core is comprised of encapsulated SAP in forms including powders, fibers or SAP in a matrix.

7. (canceled)

8. A protective article as described in claim # 1 is fabricated with biodegradable materials.

9. A protective article as described in claim # 1 is fabricated with recycled content containing materials.

10. A protective article as described in claim # 1 is fabricated with hypoallergenic materials.

11. A protective article as described in claim # 1 is fabricated with unbleached and chlorine free materials.

12. (canceled)

13. A protective cover as described in claim 1 wherein said top sheet and said back sheet joined at the outermost perimeter to encapsulate the super absorbent material by stitching, gluing, adhesive or heat sealing.

14. A protective cover as described in claim 1 wherein said back sheet is sufficiently larger in width and length than said top sheet so as to allow said back sheet to be folded up and back over outside perimeter of said top sheet and attached to said top sheet.

15. A protective article as described in claim 1 wherein said side panel is extensible.

16. A protective article as described in claim 1 wherein said top sheet is thermoplastic non-woven material.

17. A protective article as described in claim 1 where in said back sheet is thermoplastic blown film material.
